(19) United States
(12) Patent Application Publication
(10) Pub. No.: US 2008/0261299 A1
(43) Pub. Date: Oct. 23, 2008

(54) Pneumatic Bioreactor

(76) Inventors: J. Gregory Zeikus, Okemos, MI (US); Kyungnam Kim, KyungKi-Do (KR); David S. Dickey, Dayton, OH (US)

Correspondence Address: ROTHWELL, FIGG, ERNST & MANBECK, P.C., 1425 K STREET, N.W., SUITE 800, WASHINGTON, DC, 20005, US

(21) Appl. No.: 11/739,089

(22) Filed: Apr. 23, 2007

(51) Int. Cl. C12M 1/02

(52) U.S. Cl. 435/289.1

(57) ABSTRACT

A pneumatic bioreactor having a containment vessel which includes a semi-cylindrical concavity defined by the vessel bottom. A mixing apparatus includes a rotational mixer rotatably mounted within the containment vessel about a horizontal axis. The rotational mixer has buoyancy-driven mixing cavities which are fed by a gas supply beneath the rotational mixer. The mixing apparatus extends into the semi-cylindrical concavity to substantially fill that concavity. The rotational mixer is divided into two wheels with outer paddles extending axially outwardly and inner paddles extending axially inwardly on either side of each ring. Blades between the outer and inner paddles form impellers in the wheels to induce axial flow through the rings in opposite directions. The containment vessel may be of film and supported by a structural housing also having a semi-cylindrical concavity defined by the housing bottom.

### BACKGROUND OF THE INVENTION

The field of the present invention is bioreactors with mixing.

Efforts of biopharmaceutical companies to discover new biological drugs have increased exponentially during the past decade. Most biological drugs are produced by cell culture or microbial fermentation processes which require sterile bioreactors and an aseptic culture environment. However, shortages of global biomanufacturing capacity are anticipated in the foreseeable future. An increasing number of biological drug candidates are in development. Stringent testing, validation, and thorough documentation of process for each drug candidate are required by FDA to ensure consistency of the drug quality used for clinical trials to the market. Further, production needs will increase as such new drugs are introduced to the market. Bioreactors have also been used for cultivation of microbial organisms for production of various biological or chemical products in the beverage and biotechnology industries as well as for pharmaceuticals.

Stainless steel stir tanks have been the only option for large scale production of biological products in suspension culture. Manufacturing facilities with conventional stainless bioreactors, however, require large capital investments for construction, high maintenance costs, long lead times, and inflexibilities for changes in manufacturing schedules and production capacities.

A production bioreactor contains culture medium in a sterile environment that provides various nutrients required to support growth of the biological agents of interest. Conventional bioreactors use mechanically driven impellers to mix the liquid medium during cultivation. The bioreactors can be reused for the next batch of biological agents after cleaning and sterilization of the vessel. The procedure of cleaning and sterilization requires a significant amount of time and resources. The problems with sterilization are compounded by the need to monitor and to validate each cleaning step prior to reuse for production of biopharmaceutical products.

Single use disposable bioreactor systems have been introduced to market as an alternative choice for biological product production. Such devices provide more flexibility on biological product manufacturing capacity and scheduling, avoid risking major upfront capital investment, and simplify the regulatory compliance requirements by eliminating the cleaning steps between batches. However, the mixing technology of the current disposable bioreactor system has limitations in terms of scalability to sizes beyond 200 liters and the expense of large scale units. Therefore, a disposable single use bioreactor system which is scaleable beyond 1000 liters, simple to operate, and cost effective will be needed as a substitute for conventional stainless steel bioreactors for biopharmaceutical research, development, and manufacturing. While several methods of mixing liquid in disposable bioreactors have been proposed in recent years, none of them provide efficient mixing in large scale (greater than 1000 liters) without expensive operating machinery.

### SUMMARY OF THE INVENTION

The present invention is directed to a bioreactor with mixing apparatus including a rotational mixer in a containment vessel capable of efficiently and thoroughly mixing solutions without contamination. Large scale disposable units are also contemplated. The bioreactor includes a gas supply driving a rotational mixer having buoyancy driven mixing cavities.

In a first separate aspect of the present invention, the containment vessel includes a bottom defining a semi-cylindrical concavity. The mixing apparatus extends into the semi-cylindrical concavity to fill the concavity with space between the mixing apparatus and the vessel sides and bottom sufficient to avoid inhibiting free rotation of the rotational mixer. Thorough mixing of all material within the contained vessel is achieved.

In a second separate aspect of the present invention, the containment vessel includes a bottom defining a semi-cylindrical concavity. The mixing apparatus extends into the semi-cylindrical concavity to fill the concavity with space between the mixing apparatus and the vessel sides and bottom sufficient to avoid inhibiting free rotation of the rotational mixer. The rotational mixer includes two parallel wheels displaced from one another. Inner paddles on the rotational mixer are disposed to induce flow axially through each wheel in opposite directions with rotation of the rotational mixer. Patterns of flow are thus developed to enhance mixing with rotation of the rotational mixer.

In a third separate aspect of the present invention, the containment vessel includes a bottom defining a semi-cylindrical concavity. The mixing apparatus extends into the semi-cylindrical concavity to fill the concavity with space between the mixing apparatus and the vessel sides and bottom sufficient to avoid inhibiting free rotation of the rotational mixer. The rotational mixer includes two parallel wheels displaced from one another. Inner paddles on the rotational mixer are disposed to induce flow axially through each wheel in opposite directions with rotation of the rotational mixer. The rotational mixer further includes vanes disclosed to induce flow radially outwardly with rotation of the rotational mixer. The inner paddles may also induce flow radially outwardly.

In a fourth separate aspect of the present invention, the containment vessel includes a bottom defining a semi-cylindrical concavity. The mixing apparatus extends into the semi-cylindrical concavity to fill the concavity with space between the mixing apparatus and the vessel sides and bottom sufficient to avoid inhibiting free rotation of the rotational mixer. The rotational mixer includes two parallel wheels displaced from one another. Each of these wheels has two parallel plates with the buoyancy driven mixing cavities extending between the parallel plates in each wheel. The gas supply includes two orifices located below the buoyancy driven mixing cavities. The orifices may be offset to either side of the horizontal axis for rotatably mounting the rotational mixer to supply gas independently for control of rotation of the rotational mixer in opposite directions.

In a fifth separate aspect of the present invention, the containment vessel includes a bottom defining a semi-cylindrical concavity. The mixing apparatus extends into the semi-cylindrical concavity to fill the concavity with space between the mixing apparatus and the vessel sides and bottom sufficient to avoid inhibiting free rotation of the rotational mixer. The rotational mixer includes two parallel wheels displaced from one another. Each of these wheels has two parallel plates with the buoyancy driven mixing cavities extending between the parallel plates in each wheel. A structural housing including housing sides and a semi-cylindrical housing bottom into which the containment vessel is positioned. The vessel sides and the vessel bottom line the structural housing and are nonstructural film supported by the housing sides and housing bottom.

In a sixth separate aspect of the present invention, the rotational mixer further includes two parallel wheels displaced from one another and inner paddles disposed to induce flow axially through each wheel in opposite directions with rotation of the rotational mixer. Vanes disposed to induce flow radially outwardly with rotation of the rotational mixer may be included with the rotational mixer.

In a seventh separate aspect of the present invention, the rotational mixer further includes two parallel wheels displaced from one another and inner paddles disposed to induce flow axially through each wheel in opposite directions with rotation of the rotational mixer. Outer paddles disposed to mix and to induce flow axially as well with rotation of the rotational mixer are included with the rotational mixer with the inner paddles and the outer paddles being on opposite sides of the wheels. The outer paddles extend axially outwardly from the two parallel wheels and the inner paddles extend axially inwardly from the two parallel wheels.

In an eighth separate aspect of the present invention, any of the foregoing aspects are contemplated to be employed in combination to greater advantage.

Accordingly, it is a principal object of the present invention to provide an improved pneumatic bioreactor. Other and further objects and advantages will appear hereinafter.

### BRIEF DESCRIPTION OF THE DRAWINGS

FIG. 1 is a perspective view of a pneumatic bioreactor shown through a transparent housing and containment vessel for clarity.

FIG. 2 is a front view of the pneumatic bioreactor of FIG. 1.

FIG. 3 is top view of the pneumatic bioreactor of FIG. 1.

FIG. 4 is a perspective view of the top and mixing apparatus of the pneumatic bioreactor of FIG. 1.

FIG. 5 is a perspective view of one wheel of the pneumatic bioreactor of FIG. 1.

FIG. 6 is a perspective view of the top and mixing apparatus of a modified bioreactor of FIG. 1.

### DETAILED DESCRIPTION OF THE PREFERRED EMBODIMENTS

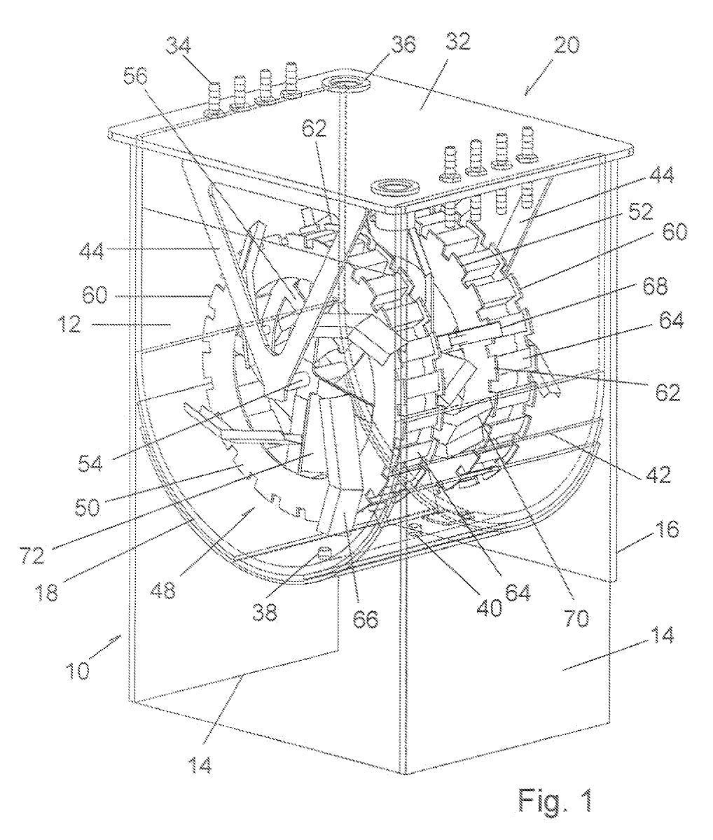

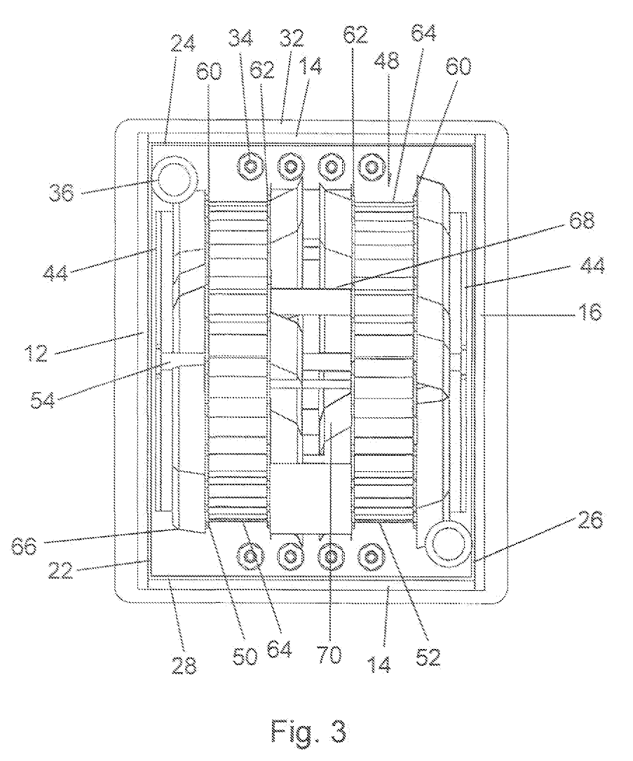

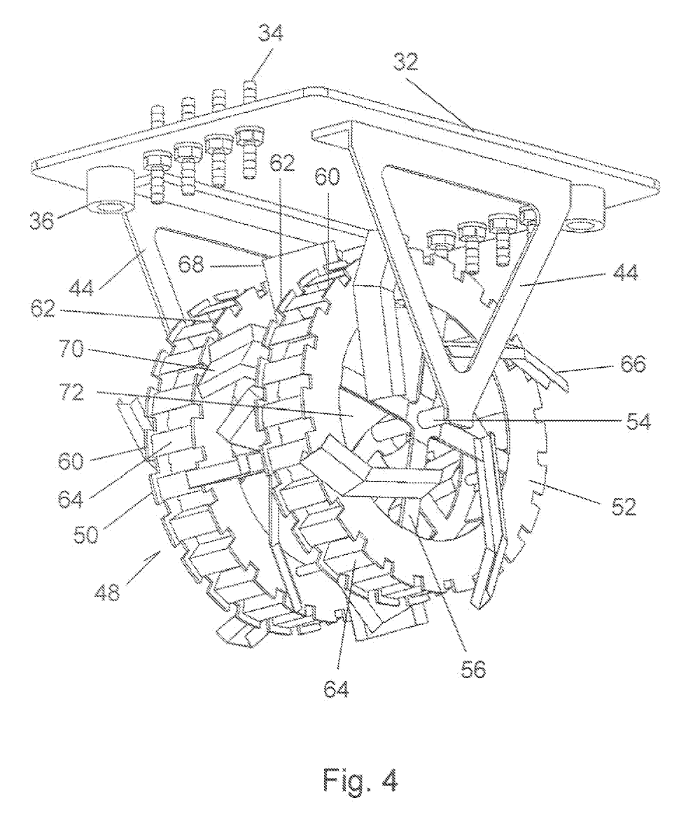

Turning in detail to the drawings FIGS. 1 through 5 illustrate a first bioreactor positioned in a housing, generally designated 10. The housing 10 is structural and preferably made of stainless steel to include a housing front 12, housing sides 14 and a housing back 16. The housing back 16 does not extend fully to the floor or other support in order that access may be had to the underside of the bioreactor. The housing 10 includes a housing bottom 18 which extends from the housing sides 14 in a semi-cylindrical curve above the base of the housing 10. One of the front 12 or back 16 may act as a door to facilitate access to the interior of the housing 10.

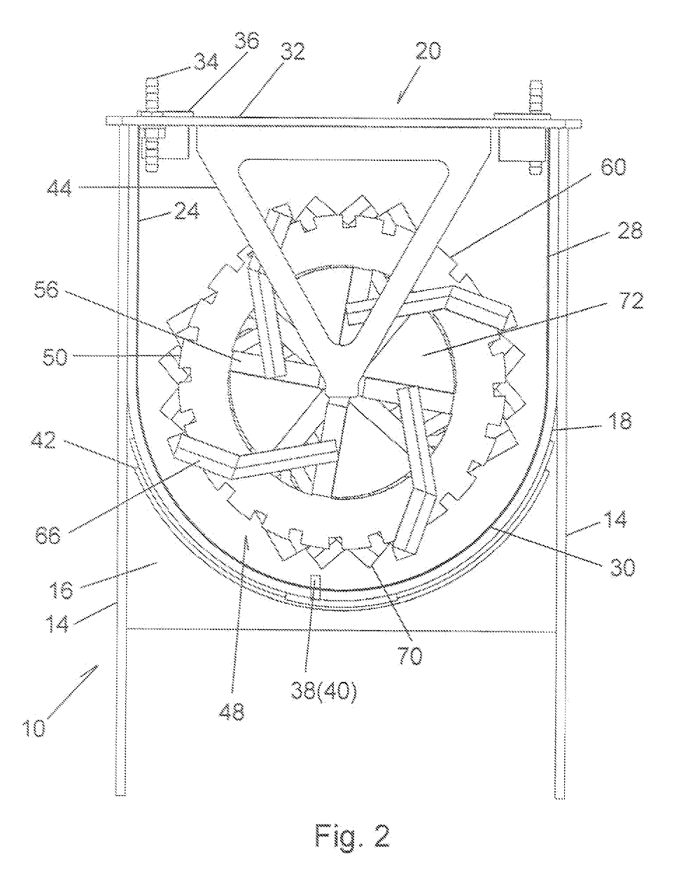

The bioreactor includes a containment vessel, generally designated 20, defined by four vessel sides 22, 24, 26, 28, a semi-cylindrical vessel bottom 30, seen in FIG. 2, and a vessel top 32. Two of the vessel sides 24, 28 which are opposed each include a semicircular end. The other two vessel sides 22, 26 join with the semi-cylindrical vessel bottom 30 to form a continuous cavity between the two vessel sides 24, 28. All four vessel sides 22, 24, 26, 28 extend to and are sealed with the vessel top 32 to form a sealed enclosure. The vessel top 32 extends outwardly of the four vessel sides 22, 24, 26, 28 so as to rest on the upper edges of the structural housing front 12, sides 14 and back 16. Thus, the containment vessel 20 hangs from the top 32 in the housing 10. The vessel, with the exception of the vessel top 32, is of thin wall film which is not structural in nature. Therefore, the housing front 12, sides 14, back 16 and bottom 18 structurally support the containment vessel 20 depending from the vessel top 32 when filled with liquid. All joints of the containment vessel 20 are welded or otherwise sealed to provide the appropriate sealed enclosure which can be sterilized and closed ready for use.

The vessel top 32 includes access ports 34 for receipt or extraction of liquids, gases and powders and grains of solid materials. The access ports 36 in the vessel top 32 provide for receipt of sensors to observe the process. Two orifices 38, 40 are shown at the vessel bottom 30 slightly offset from the centerline to receive propellant gas for driving the rotational mixer as will be discussed below. The semi-cylindrical vessel bottom 30 defining a semi-cylindrical concavity within the containment vessel 20 also includes a temperature control sheet 42 which may include a heater with heating elements, a cooler with cooling coils, or both as may be employed to raise or lower the temperature of the contents of the containment vessel 20 during use. Sealed within the enclosure defining the containment vessel 20, struts 44 extend downwardly from the vessel top 32 to define a horizontal mounting axis at or close to the axis of curvature defined by the semi-cylindrical bottom 30.

A mixing apparatus includes a rotatably mounted rotational mixer, generally designated 48. The rotational mixer 48 is a general assembly of a number of functional components. The structure of the rotational mixer 48 includes two parallel wheels 50, 52 which are displaced from one another. These wheels are tied to an axle 54 by spokes 56. Additional stabilizing bars parallel to the axle 54 may be used to rigidify the rotational mixer 48.

Each wheel 50, 52 is defined by two parallel plates 60, 62. These plates 60, 62 include buoyancy-driven mixing cavities 64 there between. These cavities 64 operate to entrap gas supplied from below the wheels 50, 52 through the gas supply at orifices 38, 40. The orifices 38, 40 are offset from being directly aligned with the horizontal axis of rotation to insure that the buoyancy-driven cavities 64 are adequately filled with gas to power the rotational mixer 48 in rotation. In the embodiment of FIGS. 1 through 5, the buoyancy-driven cavity 64 in each one of the wheels 50, 52 are similarly oriented to receive gas from the orifices 38, 40 at the same time.

Outer paddles 66 are equiangularly placed to extend axially outwardly from the outer parallel plates 60 where they are attached. These outer paddles 66 can mix the liquid between the rotational mixer 48 and either side 24, 28. The outer paddles 66 are formed in this embodiment with a concavity toward the direction of rotation of the rotational mixer 48 and are inclined toward the direction of rotation as well such that they are disposed to induce flow entrained with constituents of the mix in the vessel inwardly toward the axis for flow through each wheel 50, 52 with the rotation of the rotational mixer 48. The outer paddles 66 may exhibit an inclined orientation on each of the outer parallel plates 60 such that any induced axial flow through each wheel 50, 52 will flow toward the center of the rotational mixer 48 in opposite directions. The number of outer paddles 66 may be increased from the four shown, particularly when the constituents of the mix in the vessel are not easily maintained in suspension. The outer paddles 66 may extend close to the vessel bottom 30 to entrain constituents of the mix in the vessel which may otherwise accumulate on the bottom. Such extensions beyond the wheels 50, 52 preferably do not inhibit rotation of the rotational mixer 48 through actual or close interaction with the vessel wall.

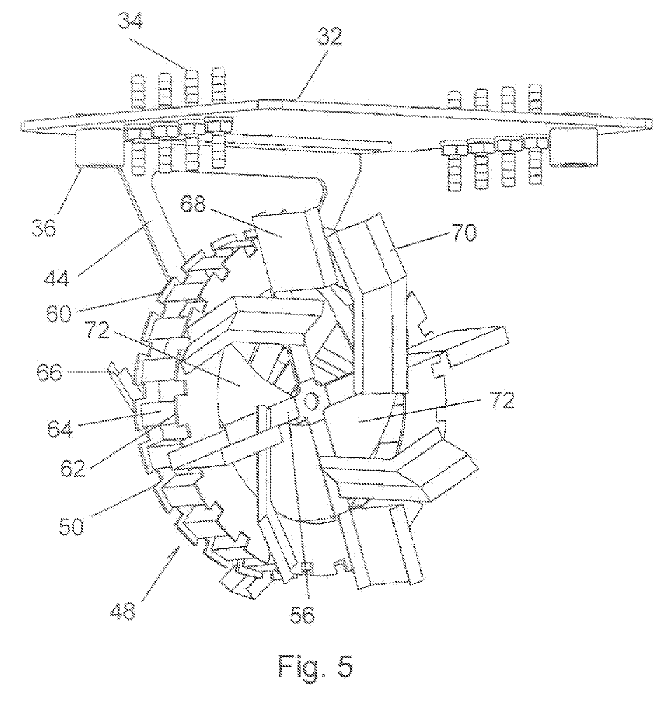

Inwardly of the two wheels 50, 52, vanes 68 may be employed in some embodiments as can best be seen in FIG. 5. These vanes 68 extend axially inwardly from the inner parallel plates 62 to span the distance there between. The vanes 68 can also extend to induce flow radially outwardly from the rotational mixer 48 and beyond the rotational mixer 48 so as to impact and mix liquid outwardly of the rotational mixer. As with the outer paddles 66, the vanes 68 can be used to entrain constituents that tend to fall and collect on the vessel bottom 30. These vanes 68 may, in some instances not be preferred because of flow resistance or disruption of circulating flow. Empirical analysis is necessary in this regard depending on such things as rotational mixer speed, liquid viscosity, space to the vessel walls and the like. Four vanes 68 are illustrated on each wheel 50, 52 but the number can, as with the outer paddles 66, be increased or decreased with the performance of the mix.

Inner paddles 70 also extend axially inwardly from the inner parallel plates 62. These inner paddles 70 are convex facing toward the rotational direction and are inclined to draw flow axially through the wheels 50, 52. The inner paddles 70 can enhance radially outward flow with rotation of the rotational mixer 48 as well at the location shown inside of the wheels 50, 52. There can be any practical number of inner paddles 70, four being shown. Such paddles 70, if configured to extend past the perimeter of the wheels 50, 52, can urge flow off of the bottom as well and direct that flow axially outwardly to either side.

Located inwardly of each wheel 50, 52 is an impeller having blades 72. The two impellers provide principal axial thrust to the flow through the wheels 50, 52. The thrust resulting from these blades 72 both flow inwardly toward one another in this embodiment. This is advantageous in creating toroidal flow about the wheels and balance forces which would otherwise be imposed on the mountings. The placement of the blades 72 may be at other axial locations such as at either of the plates 60, 62. Where the impellers act alone, the blades 72 can be located anywhere from exterior of to interior to the rotational mixer with appropriate reconfiguration in keeping with slow speed impeller practice.

The mixing apparatus defined principally by the rotating rotational mixer 48 is positioned in the containment vessel 20 such that it extends into the semi-cylindrical concavity defined by the vessel bottom 30 and is sized, with the outer paddles 66, vanes 68 and inner paddles 70, to fill the concavity but for sufficient space between the mixing apparatus and the vessel sides 24, 28 and bottom 30 to avoid inhibiting free rotation of the rotational mixer 48. In one embodiment, the full extent of the mixing apparatus 26 is on the order of 10% smaller than the width of the cavity in the containment vessel 20 and about the same ratio for the diameter of the rotational mixer 48 to the semi-cylindrical vessel bottom 30. This spacing is not critical so long as the mixing apparatus is close enough and with commensurate speed to effect mixing throughout the concavity. Obviously, empirical testing is again of value. The liquid preferably does not extend above the mixing apparatus and the volume above the rotational mixer 48 will naturally be mixed as well.

In operation, the liquid, nutrients and active elements are introduced into the containment vessel 20 through the ports 34, 36. The level of material in the vessel 20 is below the top of the rotational mixer 48 to avoid the release of driving gas under the liquid surface which may cause foam. Gas is injected through the orifices 38, 40 to become entrapped in the buoyancy-driven cavity 64 in the rotational mixer 48. This action drives the rotational mixer 48 in a direction which is seen as clockwise in FIG. 2.

The blades 72 act to circulate the liquid within the containment vessel 20 with toroidal flow in opposite directions through the wheels 50, 52, radially outwardly from between the wheels 50, 52 and then radially inwardly on the outsides of the rotational mixer 48 to again be drawn into the interior of the rotational mixer 48. Mixing with turbulence is desired and the outer paddles 66, the vanes 68 and the inner paddles 70 contribute to the mixing and to the toroidal flow about each of the wheels 50, 52. The target speed of rotation is on the order of up to the low tens of rpm to achieve the similar mixing results as prior devices at 50 to 300 rpm. The difference may reduce shear damage in more sensitive materials. Oxygen may be introduced in a conventional manner as well as part of the driving gas to be mixed fully throughout the vessel 20 under the influence of the mixing apparatus.

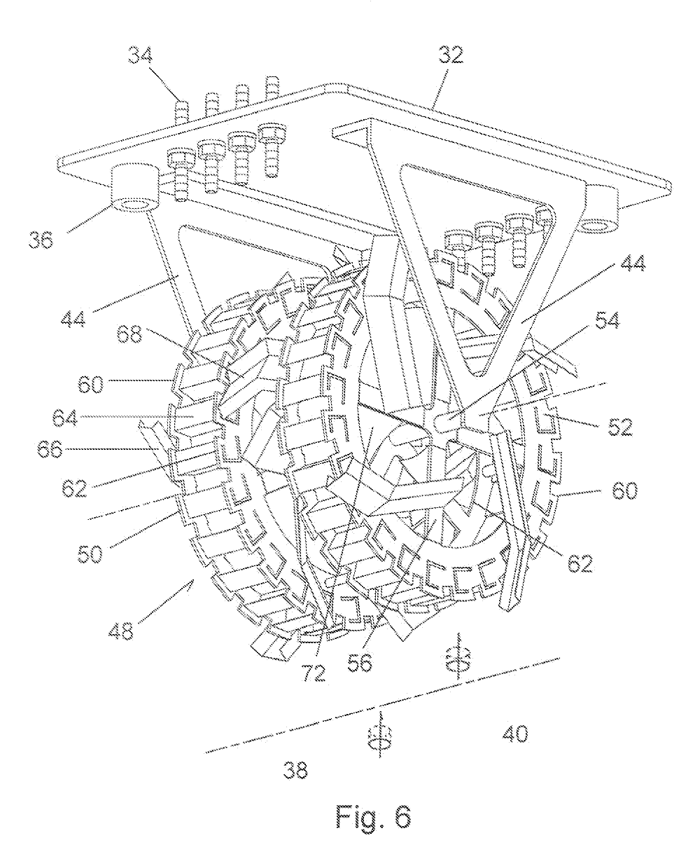

FIG. 6 illustrates a variation on the embodiment of FIGS. 1 through 5. In this embodiment, the buoyancy-driven mixing cavities 64 are reversed in one of the wheels 50, 52 for driving in the opposite direction. Similarly, the orifices 38, 40 are offset to either side of the horizontal axis of rotation. The gas through the orifices 38, 40 is independently controlled to allow selection of rotation of the rotational mixer in either direction.

Thus, an improved pneumatic bioreactor is disclosed. While embodiments and applications of this invention have been shown and described, it would be apparent to those skilled in the art that many more modifications are possible without departing from the inventive concepts herein. The invention, therefore, is not to be restricted except in the spirit of the appended claims.

What is claimed is:

1. A pneumatic bioreactor comprising
a containment vessel including vessel sides and a bottom, the bottom defining a semi-cylindrical concavity;
a gas supply having at least one orifice in the bottom of the containment vessel;
mixing apparatus including a rotational mixer rotatably mounted in the containment vessel about a horizontal axis, the rotational mixer having buoyancy-driven mixing cavities above the at least one orifice, the mixing apparatus extending into the semi-cylindrical concavity to fill the concavity with space between the mixing apparatus and the vessel sides and bottom sufficient to avoid inhibiting free rotation of the rotational mixer.

2. The pneumatic bioreactor of claim 1, the containment vessel further including a top fixed to the vessel sides, the top, the vessel sides and the bottom forming a sealed enclosure.

3. The pneumatic bioreactor of claim 2 further comprising
struts extending from the top into the containment vessel to define the horizontal axis for rotatably mounting the rotational mixer.

4. The pneumatic bioreactor of claim 2, the top including access ports therethrough.

5. The pneumatic bioreactor of claim 1, the rotational mixer further having two parallel wheels displaced from one another.

6. The pneumatic bioreactor of claim 5, the rotational mixer further having blades disposed to induce flow axially through each wheel in opposite directions with rotation of the rotational mixer.

7. The pneumatic bioreactor of claim 6, the blades defining an impeller within each wheel.

8. The pneumatic bioreactor of claim 6, the rotational mixer further having outer paddles disposed to mix and to induce radial flow with rotation of the rotational mixer, inner paddles disposed to induce flow radially outwardly with rotation of the rotational mixer, the outer paddles and the inner paddles being on opposite sides of the two parallel wheels,

9. The pneumatic bioreactor of claim 5, each of the wheels having two parallel plates, the buoyancy-driven mixing cavities extending between the parallel plates in each wheel, there being two of the at least one orifice under the buoyancy-driven mixing cavities of the wheels, respectively.

10. The pneumatic bioreactor of claim 9, the two orifices being offset to either side of the horizontal axis for rotatably mounting the rotational mixer to supply gas independently for rotation of the rotational mixer in opposite directions.

11. The pneumatic bioreactor of claim 1, the rotational mixer further having two parallel wheels displaced from one another, outer paddles extending axially outwardly from the two parallel wheels and disposed to mix and to induce flow radially inwardly, inner paddles extending axially inwardly from the two parallel wheels and disposed to induce flow radially outwardly with rotation of the rotational mixer and blades forming a impeller in each wheel to induce flow through each wheel with rotation of the rotational mixer.

12. The pneumatic bioreactor of claim 11 the rotational mixer further having vanes extending between the two parallel wheels and disposed to mix and to induce flow radially outwardly from each wheel with rotation of the rotational mixer.

13. The pneumatic bioreactor of claim 1 further comprising
a structural housing including housing sides and a semi-cylindrical housing bottom, the vessel sides and the vessel bottom lining the structural housing and being nonstructural film supported by the housing sides and housing bottom.

14. A pneumatic bioreactor comprising
a containment vessel;
a gas supply having at least one orifice in the containment vessel;
mixing apparatus including a rotational mixer rotatably mounted in the containment vessel about a horizontal axis, the rotational mixer having buoyancy-driven mixing cavities above the at least one orifice, the rotational mixer further having two parallel wheels displaced from one another and blades disposed to induce flow axially through the wheels in opposite directions with rotation of the rotational mixer.

15. The pneumatic bioreactor of claim 14, the rotational mixer further having outer paddles disposed to mix and to induce flow radially inwardly with rotation of the rotational mixer and inner paddles to induce flow radially outwardly with rotation of the rotational mixer, the outer paddles being on opposite sides of the wheels from the inner paddles.

16. The pneumatic bioreactor of claim 15, the rotational mixer further having vanes extending between the wheels disposed to mix and to induce flow radially outwardly from each wheel with rotation of the rotational mixer, the inner paddles further being disposed to induce flow radially outwardly with rotation of the rotational mixer.

17. The pneumatic bioreactor of claim 14, each of the wheels having two parallel plates, the buoyancy-driven mixing cavities extending between the parallel plates in each wheel, there being two of the at least one orifice under the buoyancy-driven mixing cavities of the wheels, respectively.

18. The pneumatic bioreactor of claim 17 the two orifices being offset to either side of the horizontal axis for rotatably mounting the rotational mixer to supply gas independently for rotation of the rotational mixer in opposite directions.
